(19) United States
(12) Patent Application Publication
(10) Pub. No.: US 2015/0025427 A1
(43) Pub. Date: Jan. 22, 2015

(54) RETRACTABLE AND ADJUSTABLE HEATING BRACE

(71) Applicant: CHIEN-CHOU CHEN, TAINAN CITY (TW)

(72) Inventor: CHIEN-CHOU CHEN, TAINAN CITY (TW)

(21) Appl. No.: 13/946,106

(22) Filed: Jul. 19, 2013

(51) Int. Cl. A61F 5/02; A61F 7/00

(52) U.S. Cl. CPC A61F 5/028; A61F 7/007
USPC 602/2

(57) ABSTRACT

The invention relates to a retractable and adjustable heating brace. Primarily, a main body is provided with a tie portion, retractable portions and assembly portions. A heating piece is disposed within the tie portion, and the assembly portion is provided with a temperature controller. Furthermore, a first conductive wire is connected to the heating piece at one end thereof and to the temperature controller at the other end thereof by extending through the first, second and third spaces, wherein the length of the first conductive wire located in the retractable portion is larger than the length of the retractable portion. Thereby when the user ties the main body around his or her waist by pulling the retractable portion thereof, the length of the first conductive wire can be stretched appropriately according to the stretch of the retractable portion. Accordingly, it can prevent the first conductive wire from break or damage.

### BACKGROUND OF THE INVENTION

1. Field of the Invention

The present invention relates to a retractable and adjustable heating brace for tying well around a user's body and providing fomentation function.

2. Description of Related Art

As for some laborers engaged in demanding jobs, they are susceptible to getting lumbar injury due to long term carrying weights by the same posture. Besides, the workers who have sedentary occupations such as word processing and so on, are also susceptible to getting lumbar injury due to poor posture. Recently, because of the prevailing leisure and sports, people usually do exercise in their leisure time. However, people sometimes get sport injury during exercise due to carelessness or over training, or sometimes twist their waists accidentally when carrying weights as usual. Therefore, if people get waist injury during work, exercise, or routine activity, they usually wear a brace on the waist for protecting the injuries. Thus, the brace can take the effect of medical treatment or health protection.

Most of conventional braces for protecting the waist are long bands, and equipped with fastening devices on both ends thereof. In use of the brace, users should wear it around the waist first, and then fasten these two fastening devices reciprocally for fixation. Certainly, when purchasing the brace, the user may choose an appropriate one depending on their waistline. However, conventional braces merely have a fixed length and lack flexibility. When users are doing exercise, the brace is unable to stretch or contract according to activities of users' bodies, so the brace may loosen or slide from users' waists. In such a case, the conventional brace may lose the effects of supporting and securing the users' injured waists.

However, the conventional braces can be used to support and secure users' injuries only and lack of the fomentation function for promoting the blood circulation of the users' injuries, so the users cannot take off the braces for a long time until recovery thereof. If the function of fomentation and the function of adjusting fomentation temperature and period according to user's demand can be further provided, the brace may facilitate the recovery of the injuries. Therefore, the conventional braces are not convenient for use during work, exercise, or routine activity.

Accordingly, in view of the above-mentioned problems, such as the lack of flexibility, fomentation, and the function of adjusting the fomentation temperature, which lead to easily loosen or slide from the users' injuries and the poor ability of assisting recovery, the present invention is to provide a retractable and adjustable heating brace.

### SUMMARY OF THE INVENTION

The present invention relates to a retractable and adjustable heating brace which can tie well around user's body and provide fomentation function.

To achieve the goal as described above, the retractable and adjustable heating brace disclosed herein comprises a main body; the main body is provided with a tie portion having a first space therein, two retractable portions having a second space respectively connected to each side of the tie portion, and a first assembly portion having a third space therein and a second assembly portion respectively connected to the other sides of the retractable portions. The first, second and third spaces are communicated with one another. A heating peace and a temperature controller are further disposed respectively within the first space of the tie portion and the third space of the first assembly portion. A temperature control button electrically connecting with the temperature controller is provided and assembled on the outside of the first assembly portion. Furthermore, a first conductive wire is connected to the heating piece at one end thereof and to the temperature controller at the other end thereof by extending through the first, second and third spaces, wherein the length of the first conductive wire located in the retractable portion is larger than the length of the retractable portion so as to stay in a curved form.

As the foregoing description, a heat insulation piece is further disposed within the first space of the tie portion of the main body and is correspondingly attached to the heating piece in proximity to the outside of the tie portion.

As the foregoing description, the inside of the first assembly portion of the main body is provided with a Velcro® hooking male part, and the outside of the second assembly portion is provided with a Velcro® fuzzy female part for correspondingly binding the Velcro® hooking male part of the first assembly portion.

As the foregoing description, the retractable and adjustable heating brace further comprises an electric power plug and a pocket disposed on the outside of the first assembly portion of the main body having a fourth space for holding the electric power plug. Moreover, the first assembly portion is provided with a through hole at a position thereof corresponding to the fourth space of the pocket for communication with the third space of the first assembly portion. A second conductive wire is provided for connection to the electric power plug at one end thereof and to the temperature controller in the third space of the first assembly portion at the other end thereof by passing through the through hole of the first assembly portion.

Accordingly, it can ensure that the main body steadily ties around a user's body by the design of the retractable portion. Furthermore, when using the heating piece for fomentation, the heat insulation piece can prevent heat evaporation from the outside of the tie portion and thus can preserve heat on the inside of the tie portion for fomentation of a user's injuries. Thereby, the invention can utilize heat effectively and increase the fomentation efficiency.

Furthermore, the length of the first conductive wire within the retractable portion is larger than the length of the retractable portion. Thereby when the user ties the main body around his or her waist by pulling the retractable portion thereof, the length of the first conductive wire can be stretched appropriately according to the stretch of the retractable portion. Accordingly, it can prevent the first conductive wire from break or damage due to a force exerted on the retractable portion for stretching by means of the reserved length for the first conductive wire as aforesaid.

### BRIEF DESCRIPTION OF THE DRAWINGS

FIG. 1 is a perspective view of the present invention.

FIG. 2 is a front view of the present invention.

FIG. 3 is a back view of the present invention.

FIG. 4 is a sectional view of the present invention.

FIG. 5 is a perspective view of the present invention in use.

### DETAILED DESCRIPTION OF THE PREFERRED EMBODIMENT

Hereinafter, an exemplary embodiment of the present invention will be described in detail with reference to the accompanying drawings.

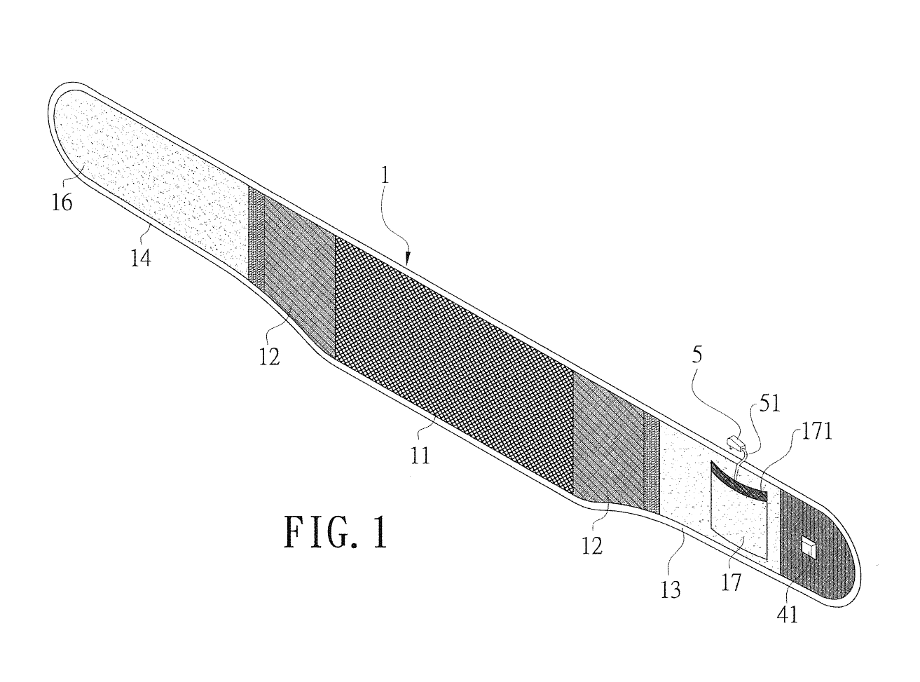

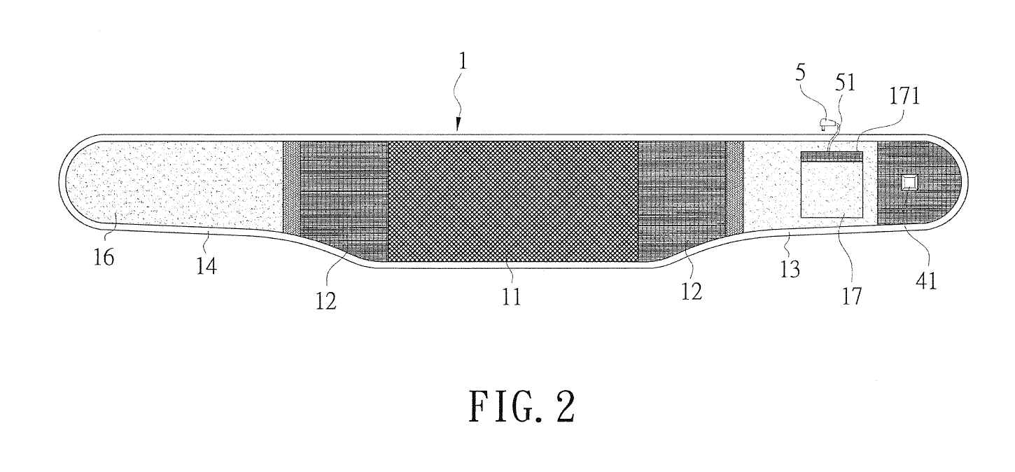

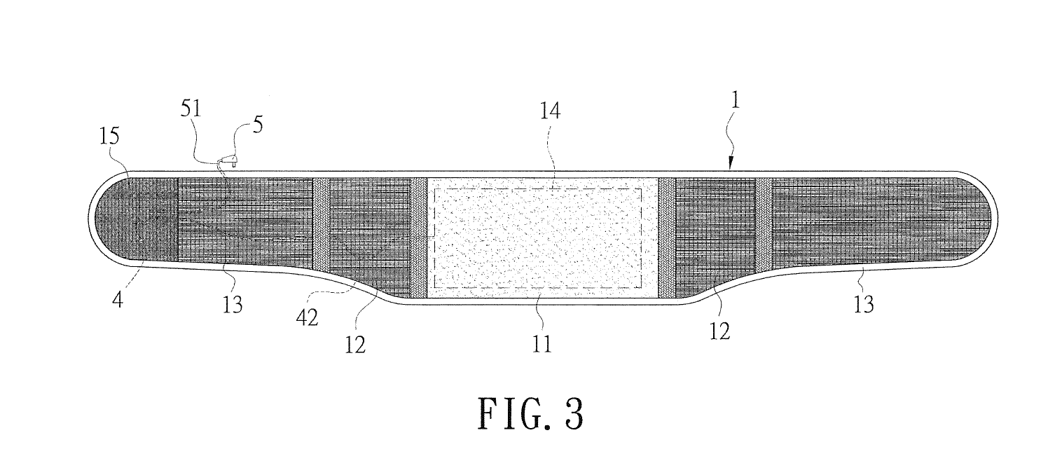

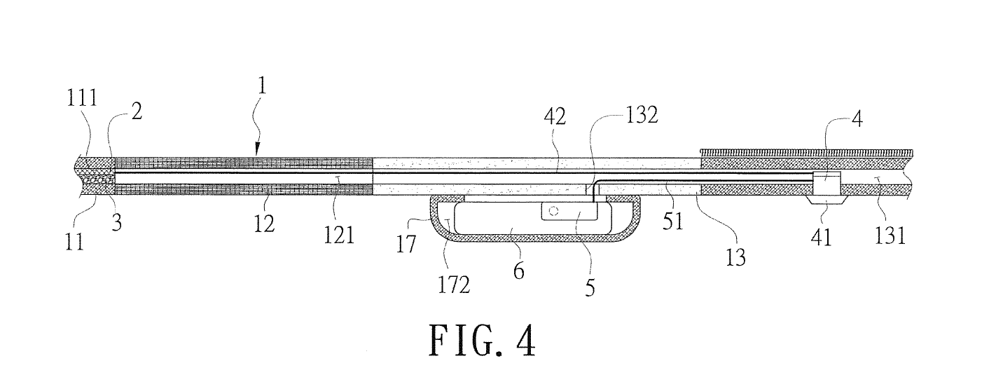

First, referring to FIG. 1 to FIG. 4, a retractable and adjustable heating brace is disclosed, comprising a main body (1), a heating piece (2), a heat insulation piece (3), a temperature controller (4), and an electric power plug (5).

The main body (1) includes a tie portion (11) having a first space (111) thereof, two retractable portions (12) having a second space (121) thereof respectively sewed on each side of the tie portion (11), and a first assembly portion (13) having a third space (131) thereof and a second assembly portion (14) respectively sewed on the other sides of the retractable portion (12), wherein the first, second and third spaces (111) (121) (131) are communicated with one another.

The inside of the first assembly portion (13) of the main body (1) is provided with a Velcro® hooking male part (15), and the outside of the second assembly portion (14) is provided with a Velcro® fuzzy female part (16) for correspondingly binding the Velcro® hooking male part (15) of the first assembly portion (13). Moreover, a pocket (17) having a fourth space (172) for holding the electric power plug (5) is disposed on the outside of the first assembly portion (13), and the opening of the pocket (17) is further disposed with a tightening belt (171).

The heating piece (2) and the heat insulation piece (3) are further disposed inside the first space (111) of the tie portion (11) of the main body (1), and the heat insulation piece (3) is correspondingly attached to the heating piece (2) in proximity to the outside of the tie portion (11).

The temperature controller (4) including a temperature control button (41) and a first conductive wire (42), and disposed inside the third space (131) of the first assembly portion (13), wherein the temperature control button (41) fitted on the outside of the first assembly portion (13) is electrically connected to the temperature control button (41), and the first conductive wire (42) is connected to the heating piece (2) at one end thereof and to the temperature controller (4) at the other end thereof by extending through the first, second and third spaces (111) (121) (131).

Furthermore, the length of the first conductive wire (42) located in the retractable portion (12) is larger than that of the retractable portion (12) and thus the conductive wire (42) stays in a curved form.

The retractable and adjustable heating brace further comprises an electric power plug (5) and a pocket (17) having a fourth space (172) for holding the electric power plug (5) disposed on the outside of the first assembly portion (13). Moreover, a second conductive wire (51) for connection to the electric power plug (5) at one end thereof and to the temperature controller (4) in the third space (131) of the first assembly portion (13) at the other end thereof by passing through a through hole (132) of the first assembly portion (13) corresponding to the fourth space (172) of the pocket (17) for communication with the third space (131) of the first assembly portion (13). Therefore, when user intends to support or secure his injuries, he can take the retractable and adjustable heating brace to tie around his injuries.

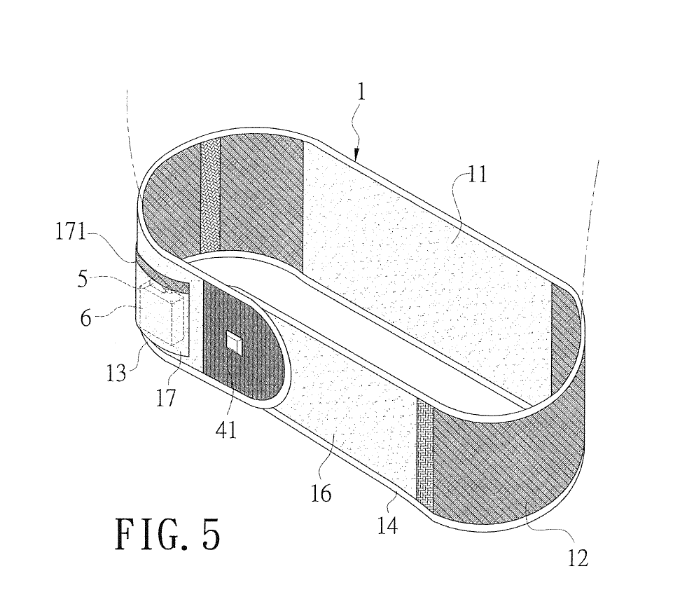

An exemplary embodiment of the present invention for the object of protecting user's waist will be described with reference to FIG. 5. Primarily, the user ties the tie portion (11) of the main body (1) on the rear waist and then fastens the first and second assembly portions (13) (14) disposed on both ends of the main body (1) from the rear waist around to the front waist. Moreover, user can let the Velcro® hooking male part (15) disposed on the inside of the first assembly portion (13) bind to the corresponding Velcro® fuzzy female part (16) disposed on the outside of the second assembly portion (14) of the main body (1). Therefore, the main body (1) of the brace can be used for tying around user's waist and provides protection and fixation for it. By the retractable portion (12) deposited on both sides of the tie portion (11) and positioned between the first and second assembly portions (13) (14), the retractable and adjustable heating brace can stretch or contract according to activities of user's body and thus can tie on user's waist well instead of loosening and sliding from it. Thereby when the user ties the main body (1) around his or her waist by pulling the retractable portion (12) thereof, the length of the first conductive wire (42) can be stretched appropriately according to the stretch of the retractable portion (12). Accordingly, it can prevent the first conductive wire (42) from break or damage due to a force exerted on the retractable portion (12) for stretching by means of the reserved length for the first conductive wire (42) as aforesaid.

Secondly, when the user intend to foment the injuries, he or she can put the battery (6) in the fourth space (172) of the pocket (17) of the main body (1), and connects the electric power plug (5) to the electric power output end of the battery (6). Then by pressing the temperature control button (41) assembled on the outside of the first assembly portion (13), the user can initiate electric current flowing from the battery (6) through the second conductive wire (51) to the heating piece (2) which further converts the electric energy into heat by the high impedance wire.

Because the heat insulation piece (3) is correspondingly attached to the heating piece (2) in proximity to the outside of the tie portion (11), when using the heating piece (2) for fomentation, the heat insulation piece (3) can prevent heat evaporation from the outside of the tie portion (11) and thus can preserve heat on the inside of the tie portion (11) for fomentation of a user's injuries. Accordingly, the heating piece (2) and the heat insulation piece (3) can increase the heating efficiency and offer well fomentation effect to reach the goals of promoting blood circulation and recovery of the user's injuries.

While the user feels the temperature of the heating piece (2) is too high or too low, he or she can adjust the temperature by pressing the temperature control button (41) again to initiate the temperature controller (4) regulating the amount of electric current flowing from the battery (6) to the heating piece (2). Meanwhile, the user can also adjust the heating period by pressing the temperature control button (41) to initiate the temperature controller (4) dominating the heating period of the heating piece (2) or stop the electric current flowing. Furthermore, from the temperature displaying unit showing different colors or numbers according to the heating temperature, the user can be informed of the heating status of the heating piece (2). Therefore, the heating temperature and period can be adjusted depending on the demand for the user's injuries to effectively assist recovery thereof.

In addition, the invention is provided with the first and second conductive wires (42) (51) within the first, second and third spaces (111) (121) (131) of the main body (1) in case of exposing outside. In this way, when the user uses, collects, or cleans the invention, the conductive wires (42) (51) are entirely protected by the main body (1) against damage. Moreover, the tightening belt (171) disposed on the opening of the pocket (17) can prevent the battery (6) placed within the pocket (17) from falling out and thus increase convenience of the usage of the present invention.

What is claimed is:

1. A retractable and adjustable heating brace comprises:
a main body including a tie portion having a first space thereof, two retractable portions having a second space thereof respectively connected to each side of the tie portion, and a first assembly portion having a third space thereof and a second assembly portion respectively connected on the other sides of the retractable portions, wherein the first, second and third spaces are communicated with one another;
a heating piece disposed inside the first space of the tie portion;
a temperature controller including a temperature control button and a first conductive wire, and disposed inside the third space of the first assembly portion, wherein the temperature control button fitted on the outside of the first assembly portion is electrically connected to the temperature control button, and the first conductive wire is connected to the heating piece at one end thereof and to the temperature controller at the other end thereof by extending through the first, second and third spaces;
wherein the length of the first conductive wire located in the retractable portion is larger than that of the retractable portion to stay in a curved form.

2. As claimed in claim 1, wherein a heat insulation piece is further disposed within the first space of the tie portion of the main body and is correspondingly attached to the heating piece in proximity to the outside of the tie portion.

3. As claimed in claim 2, wherein the inside of the first assembly portion of the main body is provided with a Velcro® hooking male part, and the outside of the second assembly portion is provided with a Velcro® fuzzy female part for correspondingly binding the Velcro® hooking male part of the first assembly portion.

4. As claimed in claim 3, wherein the retractable and adjustable heating brace further comprises an electric power plug, a pocket having a fourth space for holding the electric power plug disposed on the outside of the first assembly portion, and a second conductive wire for connection to the electric power plug at one end thereof and to the temperature controller in the third space of the first assembly portion at the other end thereof by passing through a through hole of the first assembly portion corresponding to the fourth space of the pocket for communication with the third space of the first assembly portion.
